(19) United States
(12) Patent Application Publication
(10) Pub. No.: US 2006/0254912 A1
(43) Pub. Date: Nov. 16, 2006

(54) SYSTEM AND METHOD FOR TREATING BIOLOGICAL TISSUE USIING CURRET ELECTRICAL FIELD

(76) Inventor: Amos Nussinovitch, 3 HERMAN MASS STREET (IL)

Correspondence Address: BROWDY AND NEIMARK, P.L.L.C.;624 NINTH STREET, NW, SUITE 300, WASHINGTON, DC, 20001-5303, US

(21) Appl. No.: 10/547,799

(22) PCT Filed: Mar. 4, 2004
(86) PCT No.: PCT/IL04/00223

§ 371 Date: Dec. 13, 2005

Related U.S. Application Data
(60) Provisional application No. 60/451,271, filed on Mar. 4, 2003.

(51) Int. Cl. C07K 1/26

(52) U.S. Cl. 204/450

(57) ABSTRACT

The present invention relates to a method for treating biological organic tissue, particularly plant tissue, by applying a direct current preferably of low voltage electrical field for short duration, with minimal detrimental influence on the treated tissue and to further beneficial uses of the residual tissue mass. The system and methods are useful for extraction and separation of substances of interest from the biological tissue, with optional concomitant reduction in enzyme activity, resulting in delayed or arrested browning of the tissue, and for production of imprint marks on the treated tissue. The present invention further relates to substances including pigments, minerals, oils, fragrances, vitamins, amino acids, alkaloids, extracted using the direct current treatment system, and to apparatus for performing the treatment.

### FIELD OF THE INVENTION

The present invention relates to methods and apparatus for treating intact tissue or crude tissue portions, resulting in extraction and separation of substances of interest and optionally reduction in enzyme activity and microbial count, and production of imprint marks on the treated tissue. The present invention further relates to uses of the residual tissue mass and to substances extracted using direct current applied under specific conditions to intact tissue or crude tissue portions.

### BACKGROUND OF THE INVENTION

A biological entity is made up of organic and inorganic matter in the forms of cellular and non-cellular matter, where biological cells are known as the building units of a living entity. Cellular and extracellular matter obtained from crude biological entities serves as a source of desirable organic compounds and extracts, known to be very useful as ingredients in medicine and medical care, chemical industries, food formulations and food products as well as for uses in farming and agriculture. There are many processes and steps involved in obtaining organic compounds present in crude biological matter. Upstream processing involves early stages of purification of biological matter while still in a very crude form. Currently, there are several challenges of upstream processing, among them devising purification methods and systems which are able to separate or isolate intracellular matter, and which are cost effective at the same time in order to be applied to large scale manufacturing environments. There are forms of crude biological matter which simply are not amenable to currently available inexpensive technologies of separation or isolation of cellular matter, as expensive techniques and systems need to be used. Avoiding the use of expensive techniques and systems for obtaining cellular matter from very crude biological matter translates to providing industries, which use purified biological matter as raw materials for manufacturing high performance end products such as medicines, cosmetics, and food and feed flavorings and additives, with low cost raw materials, which ultimately translates to less expensive end products. This effect is desired by both manufacturers and by consumers of the end products.

During the past few years, the food industry has increasingly preferred the use of natural pigments in many food and beverages in place of artificial coloring materials (Timberlake, 1989, BNF Nutr. Bull., 14, 113-125). The use of colorants as additives for food and drinks is a significant factor for food manufacturers and consumers alike in determining the acceptability of processed foods. Although colorants are available from natural sources, technical problems such as cost, instability, lack of availability and FDA standardization and characterization requirements limit their application (Francis, 1981, Prof. Nutr., 13, 11-14).

Color manufacturing is frequently combined with size reduction to maximize product yield. Components are extracted from plant materials for either direct consumption (for example, fruit juices) or use in subsequent processing (for example sugar, vegetable oils and naturally occurring pigments) (Fellows, 1990, Food Processing Technology, New York: Ellis Horwood Limited.). These materials are located within the cell structure of the plants and it is necessary to disrupt the cells in order to release them.

Extraction is a general type of separation process based on selectively extracting, removing, or drawing out a target substance from a matrix of several substances by chemical, thermal, or mechanical means such as by solvents separation in liquid-liquid extraction, distillation, evaporation, or cold pressing. Different methods, processes, systems, and apparatus exist for performing extractions.

A commonly used method of liquid-liquid extraction for isolating organic compounds such as phenols, oils, and fats, involves using polar or non-polar organic solvents such as alcohol, isopropanol, acetone, phenol or DMSO as an extracting agent by selectively dissolving target substances. Methods and systems of liquid-liquid extractions have the disadvantages of involving relatively hazardous and costly organic solvents. It would be desirable to eliminate, or at least minimize, quantities of such solvents required for liquid-liquid extraction of organic substances.

Cold pressing is another commonly used method of extracting cellular matter from crude biological matter, which involves subjecting crude biological matter to high pressures, and mechanically pressing out the cellular matter from the crude biological matter. The pressed out cellular matter extract is separated from the remaining crude biological matter matrix, collected, and filtered in order to obtain target substances such as useful organic compounds. Methods of cold pressing usually result in low yields of the target substances, especially in cases where the target substances are not readily pressed out or mechanically separated from crude biological matrices.

There is thus a need for, and it would be useful to have, a process and system for electrical extraction of substances from biological matter, preferably intact tissue or crude tissue portions and to obtain intra- and extra-cellular matter substances by implementing such a process and system, in a cost effective manner.

Iontophoresis is one method that has been explored as a way to effectuate transport of agents across a tissue. Such methods have been used primarily to deliver rather than extract agents through a tissue into the body (e.g., transdermal delivery of a drug). Iontophoresis is characterized by the application of an electrical current to enhance transport across a tissue by driving ionized agents through the membranes as a result of a direct electrical field effect (e.g., electrophoresis), electroosmosis, or through electrically induced pore formation (electroporation). In practice, iontophoretic methods generally involve positioning an electrode that includes some type of reservoir on the tissue through which delivery is to occur. The reservoir typically includes a solution or an absorbent pad that contains the substance to be transferred. This is called the active or drug electrode. Another electrode is also placed in contact with the tissue to allow for the completion of the electrical circuit. This is called the return, inactive, or indifferent electrode.

Various electrical extraction strategies have been tested, including the use of different wave-forms and pulsed direct current (DC) signals rather than constant-current signals. It has been suggested that the use of pulsed DC signals should theoretically provide improved performance by allowing skin capacitance to discharge, thereby allowing for more controlled current flow and agent transport. However, many DC pulsed methods suffer from at least some of the same general problems as the constant-current DC methods.

The following U.S. patents are illustrative of general pulsed DC methods: U.S. Pat. No. 5,391,195 to Van Groningen; U.S. Pat. No. 4,931,046 to Newman; and U.S. Pat. No. 5,042,975 to Chien et al. Certain DC methods employ a combination of pulsed and continuous electric fields (see, e.g., U.S. Pat. No. 5,968,006 to Hofmann). Each of the foregoing patents, however, are limited in that they discuss only methods for delivering substances across a tissue into the body of an individual. These patents include no discussion of methods for extracting compounds from a body across a tissue.

A limited number of patents discuss certain methods of using iontophoresis in extraction of a substance from the body of an individual across a tissue. U.S. Pat. No. 5,019,034 to Weaver et al. discusses methods that utilize a series of short DC pulses to induce electroporation, in particular a state referred to as reversible electrical breakdown. Various forces can then be utilized to effectuate extraction of a substance across a tissue. Once electroporation is established, the nature of the DC pulses (e.g., pulse duration, shape and frequency) is maintained until transfer is complete. U.S. Pat. Nos. 5,730,714 and 5,362,307 to Guy et al. and U.S. Pat. No. 5,279,543 to Glikfeld et al. discuss methods for extracting and delivering substances by iontophoresis utilizing an apparatus characterized by a particular electrode arrangement. U.S. Pat. Nos. 5,771,890 and 6,023,629 to Tamada discuss particular methods in which the direction of a direct current is periodically reversed during sampling of a substance. The frequency of current reversal discussed in the '890 and '629 patents is typically very low, tending to fall within the range of 1 cycle per 20 seconds to about 1 cycle per 4 hours. The methods discussed by Guy et al. and Glikfeld et al. are limited to DC methods and Weaver et al. discuss only DC pulse methods. As with all the foregoing patents and publications, Weaver et al., Guy et al., Glikfeld et al. nor Tamada discuss the use of an AC signal to maintain a substantially constant electrical state.

U.S. Pat. No. 6,344,349 discloses a process and system for electrical extraction of intracellular matter into surrounding conductivity fluid using cycles of pulses and pauses. U.S. Pat. No. 6,496,728 discloses methods for extracting substances using alternating current. Neither of these disclosures teaches or suggests the use of electrical extraction on intact or crude tissue.

The inventors of the present application have described (Zvitov, R., and Nussinovitch, A. 2001, Biotechnol. Prog., 17, 1099-1106), the physico-chemical properties and structural changes in vegetative tissues as affected by direct current electrical field. The release of cell components from the contracted tissue was only a matter of conjecture and the industrial application of the method was neither taught nor disclosed. The use of electrical field to extract intracellular matter is well known, however, nowhere in the background art is it taught or suggested that short pulse exposure of intact biological tissue to low voltage direct current as disclosed in the present invention, my results in extraction of desirable intra- and extra-cellular tissue components with minor or negligible damage to the treated tissue, allowing its further beneficial use.

There is thus an unmet need for, and it would be highly advantageous to have a process and system for separating and obtaining tissue matter from crude and preferably intact biological matter, without detrimental effects to the residual tissue mass.

### SUMMARY OF THE INVENTION

It is an object of the present invention to provide processes, systems, and apparatus for electrical extraction of substances from biological matter, particularly plant matter. Advantageously the matter subjected to the methods of the present invention comprises intact tissue or crude tissue portions that undergo only negligible or desirable changes in its mechanical properties following the electrical exposure, thus allowing further beneficial uses of the residual tissue mass.

The present invention thus provides a method for extracting a substance of interest from intact tissue or crude tissue portions comprising the steps of:

(i) providing intact or crude tissue in a processor assembly;

(ii) subjecting the tissue to at lease one short pulse of a direct current of low voltage resulting in a tissue extract; and

(iii) collecting the tissue extract.

According to certain embodiments the present invention further provides the following additional steps:

(iv) removing the treated tissue; and optionally

(v) using the treated tissue.

According to one embodiment of the present invention the electrical extraction process comprises application of one short pulse of a direct current of low voltage. According to a specific embodiment the electrical extraction is of sufficiently short duration to avoid significant temperature elevation, therefore undesired processes such as oxidation, reduction and enzymatic activity are eliminated, the extracted material is not exposed to elevated heat and damage to the obtained substances is minimized.

According to certain embodiments enzymatic reaction is inhibited during the electrical extraction, thus the browning reaction of the tissue is reduced, delayed or arrested. According to another embodiment microorganisms present in the treated tissue, are eliminated during the electrical process. According to yet another embodiment of the present invention reduction of the specimen weight is controllable thus enabling weight loss for subsequent drying processes, if desired.

According to certain embodiments the treated tissue and the extracted material do not gain more than an average of about 0.1° C. above their initial temperature following the extraction. According to yet other embodiments a temperature increase of up to about 4.5° C. may occur with no significant changes in the mechanical properties of the treated tissue or of the extracted material.

According to yet another embodiment the treated specimen is a solid, semi-solid or a gel-like tissue and the electrical extraction is performed using direct contact between the specimen and the electrodes.

According to one embodiment the tissue is provided in a solution or medium, while according to a specific embodiment, the extraction is devoid of a conductive liquid. According to a specific embodiment, the extraction is performed in a hydrophobic medium, in which the treated tissue is placed during electrification.

According to another specific embodiment the tissue is surrounded by or sandwiched between gel layers, whereby pigments and other constituents characterized in different charges are separated during the electrical extraction operation, and accumulated within the gel. According to certain embodiments wherein the extracted material is retained within a surrounding gel, the gel comprising the extract can subsequently be used as is, dried, or further concentrated.

According to a specific embodiment the individual tissue sample(s) subjected to the electric field has a weight of about 0.02 g to about 10 g. According to another embodiment the individual tissue sample(s) subjected to the electric field has a width or a Feret diameter size of about 1 mm to about 50 mm.

The tissue or tissue portions according to the present invention may be of any shape. According to certain embodiments, at least one tissue sample comprises a predetermined shape (e.g., cube, sphere, rectangle etc.), while according to other embodiments the treated tissue comprises irregularly shaped or amorphous crude material.

According to one embodiment the extraction is performed using electrodes comprising a material selected from the group consisting of: platinum, stainless steel, carbon, and gold. According to a certain embodiment at least one of the electrodes comprises platinum.

According to another embodiment of the present invention, the treated material undergoing the extraction is left in a condition which enables its use for further purposes. According to certain embodiment further uses of the treated matter include but are not limited to food additive, cosmetics and fertilization. According to another embodiment of the present invention the treated matter is a plant tissue. According to specific embodiments the plant tissue is selected from the group consisting of: cut tissues of fruits and tubers (such as Beta vulgaris, Solanum tuberosum, Musa Xparadisiaca), whole, intact plant tissues such as seeds (for example Bixa orellana L.), small fruits (such as Smilax. aspera, and Solanum sinaicum L.), small whole leaves (e.g. Commelina communis L.) and cotyledons (such as Cucumis sativus L., Phaseolus vulgaris L.), petals (for example Dianthus caryophyllus L.), or intact juice cells of fruits (such as citrus and pomegranate).

It is another object of the present invention to provide a method for marking biological tissues or tissue fragments following an electrical pulse of short duration direct current. According to a specific embodiment the electrification process produces imprints on the surface of the electrified gel or semi-solid tissue. The imprint, comprising the shape of the electrode used, is visible or more pronounced after drying the tissue and may be used for marking the products or for aesthetic purposes. It is yet a further object of the present invention to provide an extract of intracellular and extracellular matter obtained from intact tissue or crude tissue portions following a short electrical pulse of direct current. According to specific embodiments the extracts are pigments and minerals of plant origin.

According to another object of the present invention substances extracted from plant material such as roots, stems, peels, seeds, fruits, flowers and the like are collected. According to certain embodiments the substances extracted according to the methods of the present invention comprise pigments or minerals.

According to one embodiment the extracted substances are water-soluble. According to yet another embodiment the extracted substances are not soluble in water (e.g. oils, carotenoids, betalaines, chlorophylls, or flavenoids).

Thus, according to the present invention, there is provided a process for electrical extraction of substances from intact tissue or crude tissue portions, the process comprising the step of subjecting intact or cut tissue to at least one short pulse of low voltage direct electrical current, thereby releasing the intracellular and extracellular matter from the tissue to the surrounding environment, medium or gel layer(s), and enabling the collection of said substances of interest.

According to the certain embodiments of the present invention, the direct current applied to the tissue during the extraction process is characterized by at least one of the following parameters: (i) electrical current is in a range of between about 0.001 to about 0.2 A; (ii) field strength of the electrical current is in a range of between about 0.001 to about 5 kV/cm; (iii) duration of the electrical exposure is in a range of between about 0.001 to about 600 seconds.

According to another embodiment higher current are used for shortened pulse times, while according to yet another embodiments lower current (of about 0.001-0.01 A), is applied for longer duration.

According to another aspect of the present invention, there is provided an apparatus for electrical extraction of substances from intact tissue or crude tissue portions, the apparatus comprising a processor assembly including at least one processor unit having a mechanism for electrifying the tissue by transmitting direct electrical current, such that the substances are released from the tissue to the surrounding volume.

According to a specific embodiment an apparatus for continuous electrical extraction of at least one substance of interest from intact tissue or crude tissue samples is provided comprising:
i) a conveyor capable of carrying a plurality of intact tissues or cut tissue portions;
ii) a container having low impedance into which the tissues are directed;
iii) a clutch having low impedance capable of compressing the tissues within the container and closing the electrical circuit;
iv) a DC current supplier connected to at least one component selected from the container and the clutch; and
v) at least one pump capable of retrieving the extracted substance and any accumulated liquid.

According to certain embodiments the low impedance components will be a metallic material, having high conductivity as is known in the art. According to certain embodiments the container will further comprise conducting or isolating fluid at the site of the electrification. According to yet further embodiments the pump will replenish conducting or isolating fluid at the site of the electrification or supplying new medium thereto. According to still further embodiments the conveyor will transport a large number of tissues or tissue portions per unit time.

These and further embodiments will be better understood in conjunction with the figures, description and claims that follow.

### BRIEF DESCRIPTION OF THE DRAWINGS

The invention will better be understood in relation to the drawings and detailed description of the preferred embodiments which follow:

FIG. 1: is a schematic illustration of a custom-made apparatus used to apply a DC electrical field to batch wise tissue specimens.

FIG. 2: demonstrates the optical density (OD) values at 535 nm of Beta vulgaris immersion solutions vs. time of electrical field and cryogenic freezing application. OD values of the immersion solutions of the untreated specimens were zero at all times.

FIG. 3: shows SEM micrographs (low-vacuum mode) of Beta vulgaris: a and b) untreated tissue and magnification, respectively; c and d) electrically treated tissue and magnification, respectively; e and f) cryogenically frozen tissue and magnification, respectively.

FIG. 4: presents A) typical corrected tensile stress vs. Hencky's strain relationships for untreated, electrically treated (15 V for 20 s) and cryogenically frozen (20 s) Beta vulgaris cylinders; B) typical stress-strain relationships during a single compression-decompression cycle for the same tissues as in A).

FIG. 5: is a schematic illustration of custom made apparatus for electrical treatment of small quantities of intact tissue.

FIG. 6: depicts scanning electron microscopy (SEM) micrographs (high-vacuum mode) of: A and B) untreated and electrically contracted Cucumis sativus hypocotyls, respectively; C and D) untreated and electrically contracted Phaseolus vulgaris radicles, respectively; E and F) untreated and electrically contracted (by spiral wire anode) Cucumis sativus cotyledons (abaxial-side up), respectively; G and H) untreated and electrically contracted (by spiral wire anode) Cucumis sativus cotyledons (adaxial-side up), respectively.

FIG. 7: SEM micrographs of potato specimens: A and B) spiral shape produced on specimen surface as a result of spiral anode shape and magnification.

FIG. 8: demonstrates viable bacterial counts (log cells/bead) taken before and after electrical treatment (A. 20 V for 5-30 s and B. 10 s at 2-30 V) of fluorescent E. coli bacteria encapsulated within alginate gel beads.

FIG. 9: Photomicrographs of Commelina communis epidermal peel (abaxial-side up): A) untreated tissue (closed stomata); B) electrically contracted (5 V) tissue (open stomata); C) electrically contracted (20 V) tissue (open stomata). (Bar=20 μm).

FIG. 10: light values (L*) of potato specimens after electrical application, immersion in water or untreated, as indication for the extent of browning.

### DETAILED DESCRIPTION OF THE INVENTION

Disclosures in the background art relate to numerous methods for extracting of substances from biological tissues. These disclosures are directed to treatment of processed tissue, to use of different extraction conditions and/or to extraction which results in destruction of the treated tissue. The present invention discloses for the first time electrical extraction of substances from intact tissue or crude tissue portions applying short pulse of low voltage direct current, and leaving the treated tissue with negligible changes in its mechanical properties allowing further valuable uses. In addition, it facilitates the option of retaining the extracted material within a gel that is attached to, juxtaposed with, or adjacent to the biological tissue prior to its electrification.

The present invention is based on part on the unexpected discovery that exposure of an intact tissue or crude tissue portions to direct current electrical filed under specific conditions disclosed herein, results in the release of intracellular and extracellular substances to the surrounding environment or medium, with minimal effect on the treated tissue. The extraction conditions may be controlled and modified to achieve desirable amount of the released material and desirable properties of the treated tissue, such as reduction of size to maximize product yield and removal of NaCl to achieve low-salt product.

Examples of crude biological matter that may be used according to the present invention include plant matter such as flowers, roots, stems, peels, seeds, fruits and the like; and animal solids such as mammal, fish or poultry solid wastes, for example, internal organs or giblets having substantial amounts of fats and oils, derived from growing farms or processing facilities. Preferably, the biological matter is used raw or intact but use of cut, dried, extracted or powdered tissue is also within the scope of the present invention.

The methods disclosed in the present invention achieve controlled and predictable transport of substances across tissues. In particular, the methods are used for the extraction of one or more substances from plant tissue. The methods are based in part upon the recognition that a short pulse of low voltage DC signal can be utilized to maintain the permeability of the tissue within the region such that pore size, pore density and surface charge density within the treated area. The process of applying an electrical signal to increase tissue permeability (e.g., to create or enlarge pores within the tissue) is referred to as electroporation, and the degree of permeability so obtained referred to as a state of electroporation.

Non-destructive methods for extraction of substances, particularly color, could be important for many scientific fields where detection of unique constituents is necessary, in cases where identification of ingredients that are prone to oxidation or browning is required, and if using engineering skills, the process could be scaled-up. The non-destructive method leaves the user with edible material (such as vegetable) that can go through further sugar, color, aroma and acid diffusion for baking purposes, or can structurally mimic fruit pieces. Such a process depends on the extractor's skill in adding and diffusing the necessary blend of flavors, synthetic colors and aroma material into the tissue. Thus, both pigments and the moieties remaining after extraction are produced, the latter for inclusion in the drying industry for soups, as fillers for the baking industry, or as sweetened snacks. In addition, changing the color and taste of a tissue is easier if a desirable texture exists. Moreover, since the shape can be important for a particular product, a predetermined category of shapes including irregularly shaped or amorphous material, could be cut from the biological tissue prior to its electrification and further processing.

According to the present invention electrically induced extraction of naturally occurring pigments, and other ingredients, can be achieved with only minimal or no damage to the texture and structure of the treated vegetative tissue. Although the pigment extraction is not complete (i.e. more color could be extracted), the intact tissue with reduced soluble solids, minerals and pigment content can still be utilized in other food applications (for baking, filling, etc.), whereas ground tissue, as is commonly performed, is of almost no use in food products.

The electrification according to the present invention may be induced in fluid with different ionic strengths (0-1.5M), conductivities (0.1-100 μS/Cm), pH (2-12) and composition (hydrophilic vs. hydrophobic solutions, organic vs. non organic, etc.), or applied directly to the tissue without being immersed in a fluid.

According to the present invention electric fields of high intensity are similar or equivalent to low intensities for longer times. It should be understood that when very high field strength (of about 0.5-5 kV/cm) is applied to the tissue a very short pulse duration (of about 0.001-10 seconds) is required. Alternatively, longer exposure duration (of about 10-600 seconds), is used with low field strength (of about 0.001-0.5 kV/cm).

The porosity of the treated sample can be increased by the electrical extraction and could be controlled by changing the electrode shape and size, and the electric field factors, such as time of operation and intensity of electrical field.

The extracted material optionally comprises a single substance or a mixture or a complex of different substances.

Working with thinner tissue, a larger surface electrode, and higher DC electrical fields (that still do not induce heating of the tissue) could intensify the pigment extraction.

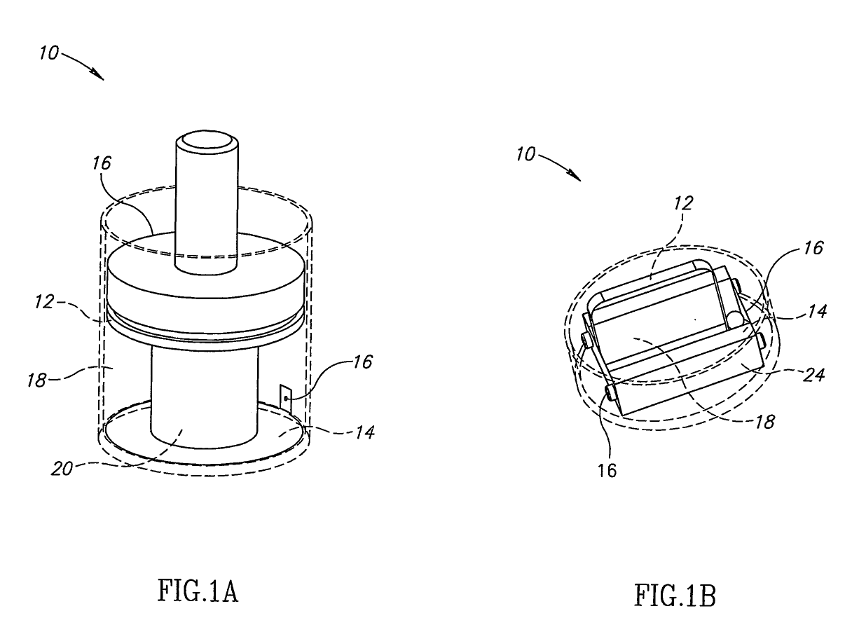

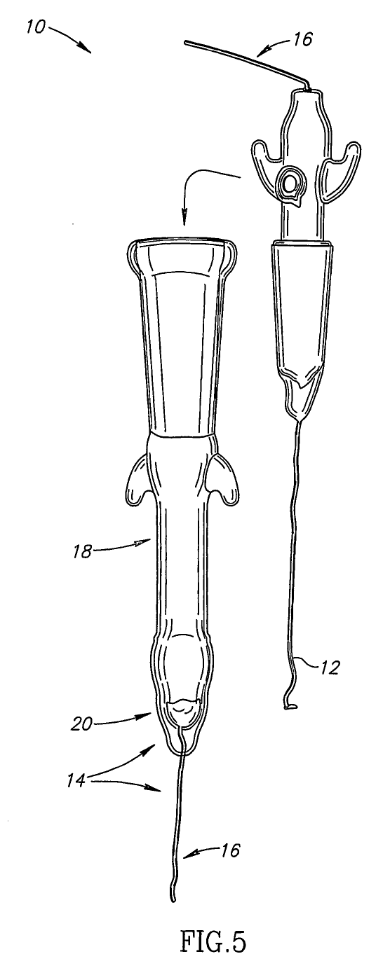

Reference is now made to FIGS. 1A, 1B, and 5 which represent schematic illustrations of custom-made apparatus, generally designated 10, used to apply a DC electrical field to tissue specimens according to certain embodiments of the invention. FIGS. 1A and 1B represent apparatus used for processing material in batches that may contain individual or multiple samples. FIG. 1A represents an exemplary apparatus for cylindrical specimens and FIG. 1B represents an exemplary apparatus for circular specimens. FIG. 5 describes a processor used for treatment of small quantities of tissue, or for laboratory scale production.

The apparatus 10 comprises an anode 12, a cathode 14, and means, for connecting to a DC power supply, 16. The apparatus 10 optionally comprises a solution 12, in which the specimen 20 is placed. The apparatus described in FIG. 1B optionally further comprises a gel component 24, used to retain the extracted material. The gel may be subsequently used as is, dried, or further concentrated.

The anode (12) and cathode (14) comprise a material selected from the group consisting of: platinum, stainless steel, carbon, and gold. According to a certain embodiment at least one of the electrodes is a platinum electrode.

The Advantages of the Method of the Invention Over Known Methods:

The substances of interest, extracted according to the present invention, can be extracted from different cellular organelles such as vacuoles and plastids (chromoplasts and chloroplasts), or from location out or between the tissue cells.

The process of the present invention facilitates extraction of both water soluble or insoluble constituents, since shrinkage of the tissue as a result of the electrification ends with an excretion of a fluid that can contain both hydrophilic and hydrophobic constituents.

The extraction is a non-thermal method by which the sample is not heated substantially during the operation.

The electrical treatment has negligible influences on the sample texture (i.e. strength, stiffness, brittleness, elasticity), therefore the treated tissue could be used for further industrial processing such as confection industry, cake fillers, baking or for other food industries.

The extraction process can lead, if desired, to volume/weight shrinkage of the treated sample, as requested, depending on time and intensity of the electrical treatment.

The electrical treatment diminishes the number of microorganisms (up to 100%) on the cut or whole tissue.

The treated tissue may be of any shape (e.g., cube, sphere, rectangle etc.) or even irregularly shaped or amorphous material.

Pigments and other constituents characterized in different charges may be separated during the electrical extraction operation and accumulated, when the treated tissue is surrounded by or sandwiched between gel layers. Wherein the extracted material is retained within a surrounding gel, the gel comprising the extract can subsequently be used as is, dried, or further concentrated.

The electrification can be applied to many tissue portions at a time.

The tissue after the electrical treatment will be with less sodium, thus more important to be consumed by those suffering from high blood pressure.

The electrical treatment on leaves can lead to stomatal opening.

The electrical treatment can inactivate or reduce the activity of different enzymes including those responsible for browning at cut surfaces.

Terminology and Definitions:

The term “extraction ” relates to removal of a material or of several materials from a mixture of substances, either for direct consumption or for use is subsequent processing (P. J. Fellowes, Food Processing Technology, CRC, Woodhead Publishing Limited, 2000).

The term “direct current” in the context of the present invention preferably means a flow of electrons only in one direction in an electric circuit (in contrast to alternating current characterized in oscillating flow of current).

As used herein, “biological tissue” includes reference to any tissue of an animal, plant, gel, fungi or algae.

As used herein and in the claims, the phrase “intact tissue” and “crude tissue portions” are used interchangeably and refer to tissue has not been subjected to substantial processing or homogenization to the point that it has lost the mechanical properties, including the texture or multicellular architecture of the original tissue. It should be clear that washed, cut, peeled, sliced, diced, segmented or immersed tissue is also within the scope of the present application.

Certain abbreviations are used herein to describe this invention and the manner of making and using it. For instance, DC refers to direct current, OD refers to optical density, SEM refers to scanning electron microscopy, TSS refers to total soluble solids,

DC electrical fields have been used to induce weight, mechanical and structural changes in alginate gels (Zvitov and Nussinovitch, 2001, Food Hydrocolloids, 15 (1), 33-42), and in agar, agarose, alginate and gellan gel beads (Zvitov and Nussinovitch, 2002, Food Hydrocolloids); physicochemical and structural changes have been induced by low DC electrical fields in vegetative tissues (Zvitov and Nussinovitch, 2001, Biotechnol. Prog., 17, 1099-1106). Since the application of the DC electrical field is very short (for tens of seconds), it was of interest to compare a treatment which takes a similar short time (cryogenic freezing) for the extraction of naturally occurring pigment. The ability to extract pigment was the outcome of another study that investigated the effects of DC electrical fields on intact plant tissues such as hypocotyls, radicles, cotyledons and leaves.

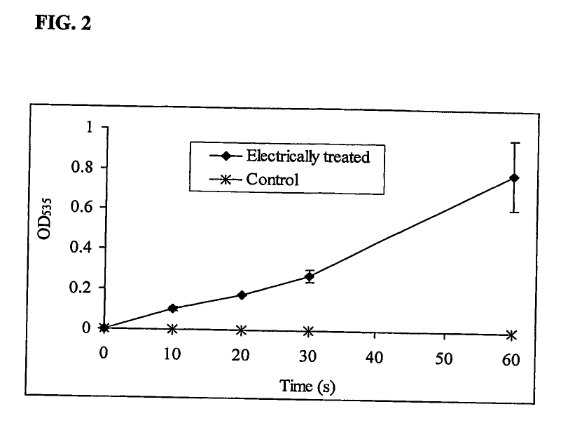

After the application of the DC electrical field or cryogenic freeze-thawing (one cycle), the presence of naturally occurring pigment was checked by spectrophotometric method. Freeze-thawing extracted more pigment than the electrical method (FIG. 2). It should, however, be noted that application of the electrical treatment to thinner slices (i.e. larger surface) or a more drastic electrical treatment could induce up to 90% color extraction from the affected vegetative beet tissue.

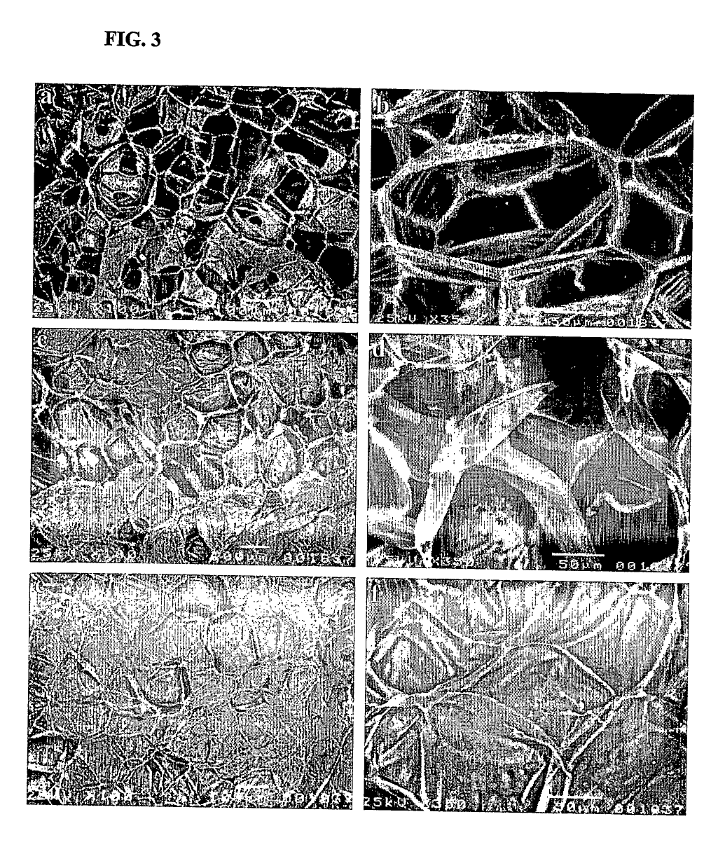

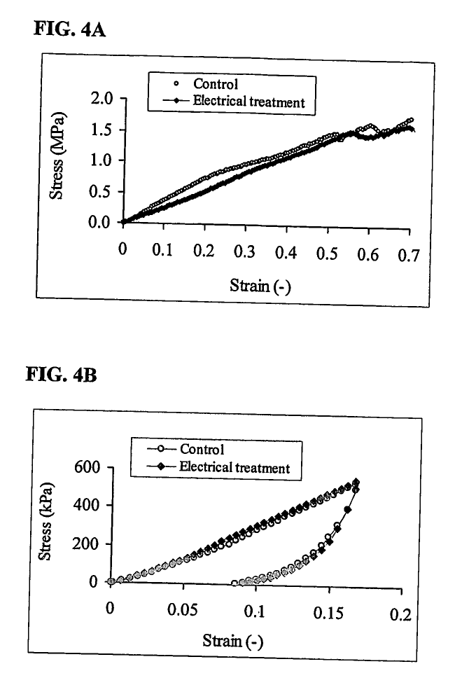

Cryogenic freezing was chosen for comparison since similar to the electrical treatment (by contacting electrodes), the cryogen is in intimate contact with the beet tissue and rapidly removes energy from it to provide its latent heat of vaporization or sublimation, to produce high heat-transfer coefficients and very rapid freezing. For reasons of comparison both treatments (electrical application and cryogenic freezing) were induced for exactly the same duration. The differences in the results were evidenced by spectrophotometric readings (FIG. 2), conductivity values, and differences in TSS (total soluble solids) and mineral composition; these differences are an outcome of the macro- and micro-textural changes that occurred in the beet tissue. Conductivity of the immersion solutions of the electrified and freeze-thawed tissues increased with time of electrical application (reaching 200 and 270 μS/cm after 60 s, respectively). The TSS of the liquid in which the tissues were immersed were: 0.0, 0.1 and 0.5 ^{o}Bx for the intact, electrified and freeze-thawed tissues, respectively (note that the ^{o}Bx for crushed tissue immersed in the same amount of water was 0.6 ^{o}Bx). The higher observed values of TSS in the freeze-thaw system were due to gross changes in the texture of the plant tissue. In addition, higher mineral content values (derived from ICP-AES analysis) of the immersion solution of the freeze-thawed tissue (88.4±0.4 mg/i) relative to those of the electrically treated (43.3±1.2 mg/l) and untreated (7.0±0.2 mg/l) tissues were observed (the content of distilled water was 2.0±0.2 mg/l). The internal stresses in the freeze-thawed tissue created by the extremely high rate of freezing caused damage to the tissue (FIG. 3e, f), although a slower freeze-thaw cycle might have caused even more intense damage to the tissue, as discussed previously (Fellows, 1990). In the electrically treated tissue (FIG. 3c, d), less cell damage and collapse were observed. To add to these results, and to strengthen the observations, the mechanical properties of the intact tissue and that treated by freeze-thawing or DC electrical field were studied (FIG. 4). The intact and DC-electrically treated tissue specimens showed and the same stress at failure (˜1500 kPa) and the same degree of elasticity (˜30%) in contrast to the tissue that passed through freeze-thawing (˜14%) and lost its elastic textural properties. In addition the average deformability modulus differed significantly and was 3000 kPa for the intact and DC-electrically treated tissue specimens vs. 130 kPa for that treated by freeze-thawing.

It is therefore disclosed that electrical fields can induce color extraction in a controllable manner, in contrast to the freeze-thaw process which is harder to control. The ability of low DC electrical fields to extract pigments with minimal harm to the treated tissue was demonstrated.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the amended claims.

The present invention is particularly exemplified hereinbelow for extraction of pigments and minerals from specific plant tissue, though this is for illustrative purposes only. Other biological tissues and other cell substances extracted using the methods disclosed are within the scope of the present invention.

### EXAMPLES

### Example 1

### Electrical Extraction Apparatus

A custom-made apparatus was built to permit electrical shrinkage of the cylindrical samples in liquid medium (FIG. 5). A few cells of similar design in different sizes were produced to permit inclusion of different-sized specimens and fluid volumes into the apparatus. The samples were sandwiched between a pair of platinum electrodes (Holland Moran LTD., Yehud, Israel) and the space was filled with distilled water. By changing the position of the electrode we could control its distance from the specimen. A DC voltage ranging from 0 to 40 V was applied across the electrodes by a DC power supply (Advice Electronics Ltd., Rosh Ha-ayen, Israel) at electrical field strength of 40 V/cm. The use of relatively low electrical field strength is desirable to minimize the absorption energy of the treated systems, and to avoid its transformation into heat.

### Example 2

### Extraction of Betalains from Red Beetroot

Betalain pigments extracted from red beetroot (Beta vulgaris) provide a natural alternative to synthetic dyes. They are derivates of betalamic acid and can be classified into two groups: the red-violet betacyanins and the yellow betaxanthins (Kujala et al., 2000, J. Agric. Food Chem., 48, 5338-5342). These water-soluble pigments are normally present at high concentrations in the vacuole of the vegetable, on the order of 1% of the total solids (Timberlake, 1989). They have been successfully used in commercial food coloring operations for a number of years (Mariassyova et al., 1999, Agri-Food Quality II: quality management of fruits and vegetables—from field to table, 314-315. Royal Society of Chemistry; Cambridge, UK). Red beetroot concentrate is universally permitted as a food ingredient, termed beetroot red (Kujala et al., 2000), although its addition to food adversely affects flavor (Francis, 1981). The pigment content in roots is from 426 to 691 mg/kg of fresh weight (Mariassyova et al., 1999). The color is usually extracted by crushing the tissue and expressing the juice (Timberlake, 1989). Different destructive methods of extraction are mentioned in the literature, including diffusion extraction in single and multiple columns and hydraulic pressing. Abeysekere and his colleagues (Abeysekere et al., 1990, J. Food Sci. Technol. Ind., 27, 336-339), demonstrated pigment yields by different extraction methods: single-column multiple extraction gave the highest relative pigment yield (98.4%) but required the most solvent. Other yields were: hydraulic pressing (88%), soaking with stirring (78%), and multiple column extraction (73%). Leakage of betalains from beet slices can also be achieved by heat shock, acid treatment, ethanol extraction and incubation in acidified 80% methanol. Putting the beet slices in deep-freeze kills them, and consequently the pigments leak out.

The novel extraction method of the present invention was compared with extraction of the same pigments by cryogenic freeze-thawing (same treatment duration), to check the methods for their efficiency on extraction of betalains and to check induced changes in both the macro- and micro-textural properties of beetroot—the vegetative source of this pigment.

Sample Preparation

Prior to specimen preparation, Fresh red beetroots (Beta vulgaris) removed from cold storage (4° C.) were allowed to equilibrate to room temperature (˜25° C.). They were peeled and halved along their longitudinal axis. Cylindrical specimens (˜200 mg), 4 mm long by 8 mm in diameter, were trimmed using a cork borer. Immediately after cutting, samples were immersed in distilled water till they reached equilibrium. Before testing, the samples were gently blotted by rolling on absorbent tissue (American Israeli Paper Mills Ltd., Hadera, Israel); their exact dimensions were determined with a digital caliper (Mitutoyo, Tokyo, Japan). Weights were measured with an analytical balance type 262SMA-FR (Precisa, Bern, Switzerland).

Cryogenic Freezing Procedure

Identical red beet specimens, immersed in distilled water at the same volume as that of the immersion solutions for the electrically treated samples, were placed in liquid nitrogen (−180° C.), using a standard receptacle.

Scanning Electron Microscopy (SEM)

The structure of B. vulgaris samples (before and after DC electrical field application) was evaluated by electron microscopy. SEM micrographs were obtained by the following procedure. The samples (before and after DC electrical field application) were mounted onto 10×10 mm SEM stubs and examined under a Jeol JSM 35C-scanning electron microscope (Tokyo, Japan) at 25 kV in low-vacuum mode (0.2 Torr).

Mechanical Testing

Uniaxial Compression

B. vulgaris samples (untreated, electrically treated with 15 V for 20 s and freeze-thawed for 20 s) were compressed between lubricated parallel plates, at a deformation rate of 10 mm/min with an Instron Universal Testing Machine (UTM), Model 5544 (Instron Co., Canton, Mass.). The UTM was interfaced to an IBM-compatible computer with a card. ‘Merlin’ software bought from the Instron Co. performed data acquisition and conversion of the UTM's continuous voltage vs. time output into digitized force-deformation, force-time, stress-strain or stress-time values with any desired definition of stress and strain. All tests were performed in at least three replicates. The deformability modulus was calculated from the linear portion of the stress-strain curve. The force vs. time data was converted to a corrected stress, σ_{cor}, vs. Hencky's strain, ε_{H}, relationship using the following substitutions (Nussinovitch et al., 1990, J. Texture Stud., 21, 37-49):

$$\begin{matrix}{σ}_{cor} = \frac{{F}_{( t )} \cdot {H}_{t}}{{A}_{o} {H}_{o}} & ( 1 ) \\ and & \\ {ɛ}_{H} = ln ( \frac{{H}_{o}}{{H}_{o} - Δ {H}_{( t )}} ) & ( 2 )\end{matrix}$$

where Ho is the original length, ΔH(t) is the momentary absolute deformation, F(t) is the momentary force at time (t), H(t) is the height of the deformed specimen, and Ao is the cross-sectional area of the original specimen.
Elasticity

B. vulgaris samples (untreated, electrically treated with 15 V for 20 s and freeze-thawed for 20 s) were subjected to a uniaxial compression-decompression cycle between two lubricated plates, as described previously (Kampf and Nussinovitch, 1997, Food Hydrocolloids, 11, 261-269). The area under the compression curve represents the total work per unit volume, whereas the area under the decompression curve is the recoverable work per unit volume. In this study, recoverable work is reported as percentage of total work. The crosshead speed was 5 mm/min. The bead was compressed by 17% (i.e. to 83% of its original height). In compression-decompression cycles, the area under the corrected stress vs. engineering strain curve was also determined by computer program using a trapezoidal method (Thomas and Finney, 1984, Calculus an Analytic Geometry, Reading, Mass.: Addision Wesley).

Conductivity Measurements

For the conductivity measurements (at 24° C.), the aforementioned red beet tissues were immersed in 5 ml distilled water. Conductivity of these solutions was determined with a Heavy Duty Conductivity/Temperature Meter, Model 407303 (Extech Instruments Co. Waltham, Mass.). The conductivity of distilled water under the same conditions was 1.0 μS/cm.

^{o}Bx Determination

For the ^{o}Bx measurements, ˜200 mg of red beet specimens (the aforementioned and crushed samples) were immersed in 3 ml distilled water. ^{o}Bx of these solutions was determined with a Palette PR-100 digital refractometer (Atago Co., Ltd. Tokyo, Japan).

Spectrophotometric Determination of Betalain Leakage

Leakage of betalain from cut cylinders (8.0 mm diameter and 4 mm height) of ˜200 mg red beet (B. vulgaris) root (after equilibrium in water) into the external fluid (4 ml distilled water) was evaluated by spectrophotometric absorption measurements in a Milton Roy Spectronic 601 spectrophotometer (Spectronic Unicam, NY) at 535 nm. The tissue cylinders were freeze-thawed (one cycle) and electrically treated with 15 V (˜40 V/cm) for various times (0-60 s); untreated tissues were examined in parallel.

Mineral Composition

For the mineral composition analysis, the aforementioned treated tissues and their untreated counterparts were immersed in 9 ml distilled water. The immersion solutions were digested in concentrated (65%) nitric acid for 7 h at 90 to 100° C. by heating in a sand bath. Analyses were conducted on aliquots of these solutions, vs. multielement standards. All elements were determined in the tested solutions by inductively coupled plasma-atomic emission spectrometry (ICP-AES systems from Spectro, Kleve, Germany). An ICP-AES instrument, model “Spectroflame modula E” from Spectro was used, with a standard cross-flow nebulizer. The power level was 1.2 kW, coolant flow 15 I/min, auxilliary flow 0.5 l/min and nebulizer flow 0.5 l/min. Concentrations are given as μg/ml of the original sample.

Statistical Analysis

In general, all statistical analyses were conducted with JMP software (SAS Institute JMP statistics and graphics guide. Version 3.1, 1995, Cary, N.C.: SAS Institute Inc.), including ANOVA and the Tukey-Kramer Honestly Significant Difference Method for comparisons of means.

### Example 3

### Extraction of Minerals from Cucumis sativus

The mineral composition of C. sativus cotyledons (untreated and electrically contracted) and of the solutions in which they were immersed, were examined. Sample batches of the leaves were digested in 5-ml volumes of nitric acid, using an MLS 1200 mega microwave digestion unit. The samples were exposed for 10 min to 500 W, and for another 10 min to 580 W of microwave radiation. The volume was brought up to 15 ml with deionized water. The solutions were diluted 1:10 with deionized water. Analyses were conducted on portions of these solutions, versus multi-element standards. All elements were determined in the tested solutions by inductively coupled plasma atomic emission spectrometry. The mineral composition of C. sativus cotyledons (untreated and electrically contracted by 10 V for 1 min) and of the solutions in which they were immersed, were examined. Higher contents of potassium, sodium, calcium and sulfur were observed as compared to control solutions (Table 1). In addition, X-ray analyses were conducted on leaves subjected to DC electrical field. Results indicated a reduction in potassium content (˜7%) compared to untreated tissues (˜25%).

TABLE 1
Concentration of K, Ca, Na and S in the immersion solutions

| | Solution concentration (mg/l) | | |
|---|---|---|---|
| | | Distilled water + leaf | Distilled water + |
| Element | Distilled water | after electrical application | leaf |
| K | 0.45 ± 0.006 | 3.4 ± 0.02 | 1.2 ± 0.007 |
| Ca | ≦0.11 | 11.9 ± 0.09 | 2.5 ± 0.05 |
| Na | 0.16 ± 0.01 | 16.4 ± 0.05 | 4.8 ± 0.001 |
| S | 0.095 ± 0.01 | 0.93 ± 0.04 | 0.27 ± 0.008 |

### Example 4

### Structural Analysis of Treated Tissues

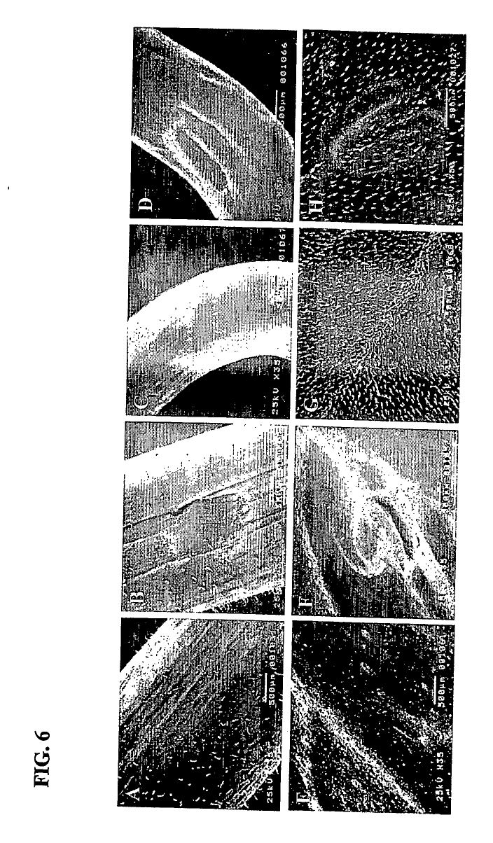

The electrical treatment can be applied to various plant tissues whether cut or intact. The structure of the plant tissues (untreated and electrically contracted by 10 V for 1 min) was evaluated by electron microscopy. A Jeol JSM 35C-scanning electron microscope was used at 25 kV in high vacuum mode (pressure of 10⁻³ mm Hg). Electron micrographs of P. vulgaris radicles, C. sativus hypocotyls and C. sativus cotyledons, before and after electrical field application, are shown in FIG. 6. In general the shape of the affected area of the shrunken tissue resembled the shape of the electrode. The shrinkage of these tissues by small DC voltages and the changes on their surface substantiated the generality of the findings.

### Example 5

### Texture Analysis

During the electrical treatment there are no significant changes in the texture of the sample treated. B. vulgaris samples (untreated, electrically treated with 15 V for 20 s) were compressed between lubricated parallel plates, at a deformation rate of 10 mm/min with an Instron Universal Testing Machine (UTM), Model 5544 (Instron Co., Canton, Mass.). The UTM was interfaced to an IBM-compatible computer with a card. ‘Merlin’ software bought from the Instron Co. performed data acquisition and conversion of the UTM's continuous voltage vs. time output into digitized force-deformation, force-time, stress-strain or stress-time values with any desired definition of stress and strain. In addition, B. vulgaris samples (untreated, electrically treated with 15 V for 20 s) were subjected to a uniaxial compression-decompression cycle between two lubricated plates (FIG. 4). The area under the compression curve represents the total work per unit volume, whereas the area under the decompression curve is the recoverable work per unit volume. In this study, recoverable work is reported as percentage of total work. The crosshead speed was 5 mm/min. The bead was compressed by 15% (i.e. to 85% of its original height). In compression-decompression cycles, the area under the corrected stress vs. engineering strain curve was also determined by computer program using a trapezoidal method. The intact and DC-electrically treated tissue specimens showed the same stress at failure (˜1500 kPa) and the same degree of elasticity (˜30%) indicating that there are no significant changes in texture during the extraction process.

### Example 6

### Porosity Changes in Treated Tissue

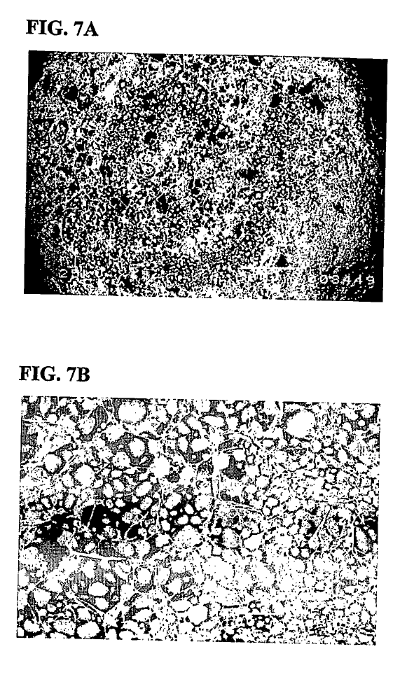

The electrical treatment can lead to higher porosity of the sample treated (claim 18). The structure of potato (before and after a DC electrical field of 20 V for 1 min) was evaluated by electron microscopy (as described in example 4). The electron micrographs of the potato specimens after the electrical treatment are shown in FIG. 7. In this study, the anode was designed as a spiral wire. The affected area of the shrunken tissue resembled the shape of the anode and so a curved spiral shape line can be seen clearly at its surface. A closer look revealed two regions within the potato: the first, which was contracted, and the second, which was contracted and had pores within the area bordered by the lines formed on the surface. The pores on the tissue can change the porosity of the plant tissue and affect its diffusivity.

### Example 7

### Reduction Microorganism Count

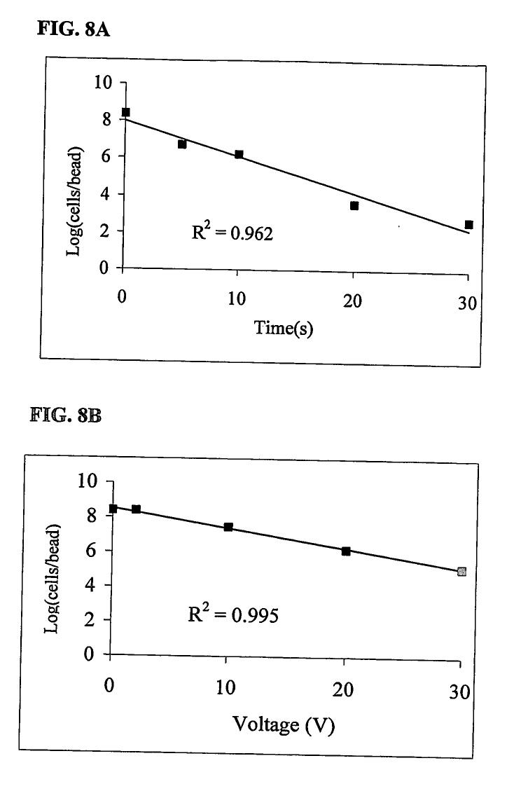

The electrical treatment can diminish the number of microorganisms (up to 100%) on the cut or whole tissue. For preliminary results we used alginate gel beads (2% alginate cross linked with 2% CaCl₂; diameter: 4 mm) as a model system for plant tissue. Fluorescent E. coli bacteria were encapsulated within the beads. Viable bacterial counts were taken before and immediately after the electrical treatment (20 V for 5-30 s and 10 s at 2-30 V). To dissolve the beads for bacterial counts, they were immersed in a 2% (w/w) sterile sodium citrate solution and vigorously shaken (400 rpm) to total dissolution (˜20 min). The released bacteria were immediately serially diluted, plated on LA amended with 50 μg/ml kanamycin at 37° C. The amount of bacterial cells was then estimated. The viability of the bacteria decreased as the time and voltage of the electrical field increased (FIG. 8).

### Example 8

### Stomatal Opening in Treated Tissue

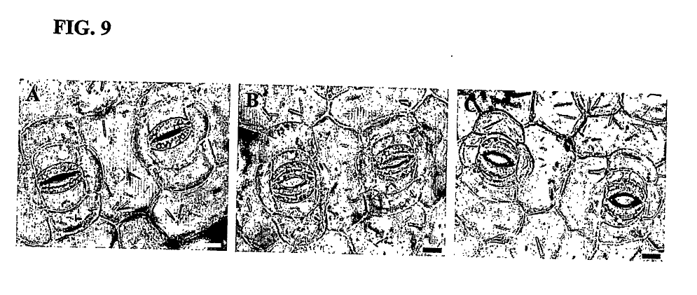

Strips of abaxial and adaxial epidermis of C. communis leaves were removed and floated on a solution containing 30 mol m⁻³ KCl, 10 mol m³ MES (pH 6.1, adjusted with KOH). The strips from the treated leaves (˜0.35 mm in height) were peeled after the electrical application (5-20 V for 10-60 s). Strips from untreated leaves were examined as well. Isolated epidermal peels of these treated leaves as well as those of the untreated tissue are shown by light microscopy (FIG. 9). Stomatal opening due to the DC electrical field can be clearly seen.

### Example 9

### Browning Reaction Reduction in Response to Electrical DC

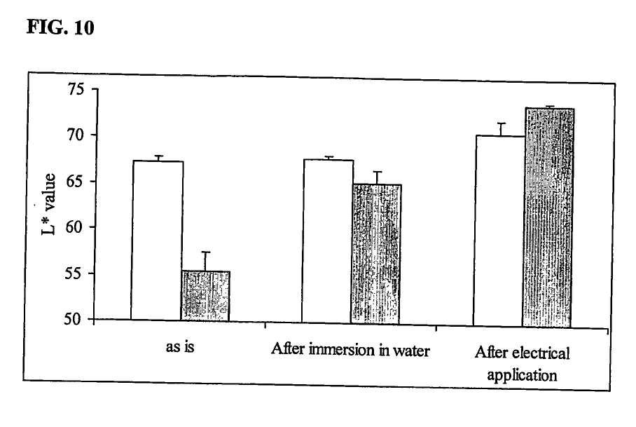

Three types of potato specimens (untreated, immersed in water for 1 min, or subjected to an electrical field intensity of 40 V/cm for 1 min) were analyzed and monitored using a Minolta Chroma Meter CR-100 (Minolta Camera Co., Ltd., Osaka, Japan). Readings are reported in the L*, a*, b* system, where L* corresponds to lightness, a* to the red/green scale and b* to the yellow/blue scale. The extent of browning in potato samples was measured by the changes in these parameters after 21 h. The L* values of potato specimens decreased after 21 h of storage at 25° C. and 60% RH (FIG. 10): the higher the L* value the lighter (less dark), the specimen. Water-dip treatment decreased the extent of browning in the potato tissue, as reported previously, and as reflected by a smaller decrease in L* value. Application of the DC electrical field resulted in the opposite effect. L* values increased and the tissue appeared lighter.

### Example 10

### Continuous Line Electrification Apparatus

In a continuous production line a large number of intact or cut tissue pieces are moved along a conveyor belt. At a predetermined location the movement is stopped and the pieces are subjected to electrification. By way of non-limitative examples the tissue or tissue pieces may be directed to a container or may simply be held or compressed between an upper metallic clutch and a lower metallic belt. The container may further contain a conductive or an isolating medium as desired. At that predetermined location a DC current is transferred through hundreds or thousands of pieces of tissue at substantially the same time.

The extracted substance(s) including any secreted liquid(s) are subsequently collected and transferred by a pump for further processing or concentration. The residual tissue matter may be discarded, or may advantageously be removed form the conveyor assembly and retained or stored until further use. Advantageously the pump may be configured to replenish a conductive or an isolating medium used for retrieving the extracted substances within the container.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

What is claimed is:

1. A method for electrically extracting substances from biological matter, wherein the biological matter comprises intact tissue or crude tissue portions that undergo negligible or desirable changes in mechanical properties as a result of an electrical exposure.

2. The method of claim 1 comprising the steps of:
(i) providing intact or crude tissue in a processor assembly;
(ii) subjecting the tissue to at lease one short pulse of a direct current of low voltage resulting in a tissue extract; and
(iii) collecting the tissue extract.

3. The method of claim 2 further comprising the steps of:
(iv) removing the treated tissue; and optionally
(v) retaining the treated tissue for further use or processing.

4. The method of claim 2 wherein the tissue is subjected to only one short pulse of a direct current of low voltage.

5. The method of any one of claims 2-4 wherein the electrical pulse is of sufficiently short duration to avoid significant temperature increase of the tissue and of the extracted material.

6. The method of claim 5 wherein the temperature increase is not more than about 0.1° C. on average.

7. The method of claim 5 wherein the temperature increase is not more than about 4.5° C. on average.

8. The method of any one of claims 1-7 resulting in elimination of oxidation, reduction or enzymatic activity in the treated tissue.

9. The method of any one of claims 8 resulting in delay, reduction or arrest of browning reaction of the treated tissue, compared to untreated tissue.

10. The method of any one of claims 1-7 resulting in elimination of or reduction of microorganisms present in the treated tissue, compared to untreated tissue.

11. The method of any one of claims 1-7 resulting in contraction of the tissue and decrease in its weight, compared to untreated tissue.

12. The method of claim 11 wherein the tissue is subsequently dried.

13. The method of any one of claims 1-12 wherein the tissue is provided in a solution or medium.

14. The method of claim 13 wherein the solution or medium comprise hydrophobic liquid.

15. The method of claim 14 wherein the solution or medium comprise oil.

16. The method of claim 13 wherein the solution or medium comprise hydrophilic liquid.

17. The method of any one of claims 1-12 wherein the tissue is provided without a conductive or isolating liquid.

18. The method of any one of the claims 1-12, wherein the tissue comprises at least one sample surrounded by or adjacent to at least one gel layer, that accumulates the extracted substances.

19. The method of claim 18, wherein the at least one gel layer that accumulates the substances is concentrated, dried or processed after the extraction.

20. The method of any one of claims 1-19 wherein the tissue comprises at least one sample having weight of about 0.02 g to about 10 g.

21. The method of any one of claims 1-19 wherein the tissue comprises at least one sample having a width or a Feret diameter size of about 1 mm to about 50 mm.

22. The method of any one of claims 1-21 wherein the extraction is performed using electrodes comprising a material selected from the group consisting of: platinum, stainless steel, carbon, and gold.

23. The method of claim 22 wherein at least one of the electrodes is a platinum electrode.

24. The method of any one of claims 1-23 wherein the biological matter is a solid, a semi-solid or a gel-like tissue and the electrical exposure is performed using direct contact between at least one tissue portion and at least one of said electrodes.

25. The method of claim 24 resulting in formation of an imprint on the treated tissue, wherein the imprint becomes stable or more pronounced after drying of the tissue.

26. The method of claim 25 wherein said imprint bears the shape of the electrode used for electrification.

27. The method of any one of claims 1-26 wherein the residual tissue is left in a condition which retains mechanical properties allowing further use of said tissue.

28. The method of claim 27 wherein the further use is selected from the group consisting of food additive, cosmetics ingredient and fertilization component.

29. The method of any one of claims 1-28 wherein the tissue is a plant tissue.

30. The method of claim 29 wherein the plant tissue is selected from the group consisting of: fruits, tubers, seeds, leaves, cotyledons, petals, and juice cells of fruits.

31. The method of claim 30 wherein the plant tissue is intact.

32. The method of claim 30 wherein the plant tissue is cut, peeled, sliced, diced or immersed.

33. The method of claim 2 wherein the direct current applied to the tissue during the extraction process is characterized by at least one parameter selected from the group consisting of:
(i) electrical current is in a range of between about 0.001 to about 0.2 A;
(ii) field strength of the electrical current is in a range of between about 0.001 to about 5 kV/cm; and
(iii) duration of the electrical exposure is in a range of between about 0.001 to about 600 seconds.

34. The method of claim 33 wherein the direct current applied to the tissue during the extraction process is characterized by at least one parameter selected from the group consisting of:
(i) the electrical current is in a range of between about 0.001 to about 0.2 A;
(ii) the field strength of the electrical current is in a range of between about 0.5 to about 5 kV/cm; and
(iii) the duration of the electrical exposure is in a range of between about 0.001 to about 10 seconds.

35. The method of claim 33 wherein the direct current applied to the tissue during the extraction process is characterized by at least one parameter selected from the group consisting of:
(i) the electrical current is in a range of between about 0.001 to about 0.2 A;
(ii) the field strength of the electrical current is in a range of between about 0.001 to about 0.5 kV/cm; and
(iii) the duration of the electrical exposure is in a range of between about 10 to about 600 seconds.

36. An extract of substances obtained from intact or crude tissue as a result of being subjected to at least one short pulse of a direct current of low voltage.

37. The extract of claim 36 produced by a method comprising the steps of:
(i) providing intact or crude tissue in a processor assembly;
(ii) subjecting the tissue to at lease one short pulse of a direct current of low voltage resulting in a tissue extract; and
(iii) collecting the tissue extract.

38. The extract of claim 37 further comprises the steps of:
(iv) removing the treated tissue; and optionally
(v) using the treated tissue.

39. The extract of any one of claims 36-38 comprising intracellular or extracellular substances.

40. The extract of any one of claims 36-39 wherein the substances are extracted from plant tissue selected from the group consisting of roots, stems, peels, seeds, fruits, flowers and the substances are collected following the extraction.

41. The extract of any one of claims 36-40 comprising at least one pigment or mineral.

42. The extract of claim 41 wherein the at least one pigment or mineral is water-soluble.

43. The extract of claim 41 wherein the at least one pigment or mineral is not soluble in water.

44. The extract of claim 43 wherein the insoluble pigment or mineral is selected from the group consisting of carotenoids, betalaines, chlorophylls, and flavenoids.

45. An apparatus for extraction of at least one substance from intact tissue or crude tissue portions wherein the extraction method comprises the steps of:
(i) providing intact or crude tissue in a processor assembly;
(ii) subjecting the tissue to at lease one short pulse of a direct current of low voltage resulting in a tissue extract; and
(iii) collecting the tissue extract.

46. The apparatus of claim 45 wherein said method further comprises the steps of:
(iv) removing the treated tissue; and optionally
(v) using the treated tissue.

47. The apparatus of claim 45 comprising a processor assembly including at least one processor unit having at least one electrode and a mechanism for electrifying the tissue by transmitting direct electrical current, such that the substance is released from the tissue and optionally collected.

48. The apparatus of claim 47 wherein at lease one of the electrodes comprises a material selected from the group consisting of platinum, stainless steel, carbon, and gold.

49. The apparatus of claim 48 wherein the at least one of the electrodes is a platinum electrodes.

50. An apparatus for continues extraction of substance of interest from intact tissue or crude tissue portions comprising:
i) a conveyor capable of carrying large number of cut tissue particles per minute;
ii) a metallic container into which the particles are directed;
iii) a metallic clutch capable of compressing the particles and closing the electrical circuit;
iv) a DC current supplier connected to any of the metallic components; and
v) at least one pump capable of transformation accumulated liquid or supplying new conducting or isolating fluid to the site of the electrification.
